(19) United States
(12) Patent Application Publication
(10) Pub. No.: US 2009/0216320 A1
(43) Pub. Date: Aug. 27, 2009

(54) Magnetic Gradient Targeting And Sequestering Of Therapeutic Formulations And Therapeutic Systems Thereof

(75) Inventors: Robert J. Levy, Merion Station, PA (US); Ivan Alferlev, Clementon, NJ (US); Michael Chorny, Philadelphia, PA (US); Ilia Fishbein, Philadelphia, PA (US); Gennady Friedman, Richboro, PA (US); Boris Polyak, Philadelphia, PA (US); Darryl Williams, Philadelphia, PA (US)

Correspondence Address: RATNERPRESTIA, P.O. BOX 980, VALLEY FORGE, PA, 19482, US

(73) Assignees: The Children's Hospital of Philadelphia, Philadelphia, PA (US); Drexel University, Philadelphia, PA (US)

(21) Appl. No.: 12/297,971

(22) PCT Filed: Apr. 20, 2007
(86) PCT No.: PCT/US07/09603

§ 371 Date: Jan. 22, 2009

Related U.S. Application Data
(60) Provisional application No. 60/794,191, filed on Apr. 21, 2006.

(51) Int. Cl. A61M 37/00; A61N 2/00; A61F 2/82

(52) U.S. Cl. 623/1.42; 600/12; 604/500

(57) ABSTRACT

A therapeutic system and a method that uses stents, and/or other implantable devices (104) for local delivery of a therapeutic agent is disclosed. A therapeutic formulation (102) may include particles of a biocompatible magnetic or magnetizable material that carry the therapeutic agent, or magnetically responsive cells. The therapeutic formulation (102) is intravenously administered to a mammalian subject. A portion of the formulation (102) is delivered to the proximity of a device (104) implanted in the vascular system of the subject by externally generating a magnetic field gradient (106) on the implantable device (104). The portion of the therapeutic formulation (102) not delivered to the proximity of the implantable device (104) is removed from the vascular system. The method allows for the repeated administration of the same or different therapeutic agent, and further, has the option of locally injecting, or alternatively, peripherally administering, the therapeutic agent.

### CROSS REFERENCE TO RELATED APPLICATIONS

This application claims priority on U.S. Provisional Patent Application 60/794,191, “Magnetic Gradient Targeting and Sequestering of Therapeutic Formulations and Therapeutic Systems Thereof,” filed Apr. 21, 2006, the disclosure of which is incorporated herein by reference.

### STATEMENT REGARDING FEDERALLY SPONSORED RESEARCH OR DEVELOPMENT

This Research was supported in part by U.S. Government funds (National Heart Lung and Blood Institute Grant No. HL72108 and NSF Grant No. 9984276), and the U.S. Government may therefore have certain rights in the invention.

### FIELD OF THE INVENTION

This invention relates to implantable devices and to methods of using the devices to target and capture therapeutic agents attached to, or encapsulated within, magnetic or magnetizable carriers within a body or a subject. In particular, the invention relates to magnetic gradient targeting of therapeutic formulations and concomitant magnetic sequestering of magnetic or magnetizable carriers during therapy via peripheral intravenous administering of magnetic or magnetizable therapeutic formulations.

### BACKGROUND OF THE INVENTION

Implantable devices, such as stents, are commonly used in a variety of biomedical applications. For example, stents are routinely implanted in patients to keep blood vessels open in the coronary arteries, to keep the esophagus from closing due to strictures of cancer, to keep the ureters open for maintenance of kidney drainage, and to keep the bile duct open in patients with pancreatic cancer. Stents typically comprise a tube made of metal or polymer, in a wide range of physiologically appropriate diameters and lengths, which are inserted into a vessel or passage to keep the lumen open and prevent closure due to a stricture or external compression.

Drug eluting stents, which consist of polymer coated metallic stents containing either taxol or sirolimus, represent a major improvement over bare metal stents. However, there is a fundamental problem with the use of drug eluting stents. They contain only one therapeutic agent, with one small dose of this agent, for one course of the administration, with no possibility for re-administration of the same or different therapeutic agent. There is no circumstance in medicine where this therapeutic approach has been a successful long term treatment for any chronic disease, such as arteriosclerosis. Furthermore, there are numerous reports of failed drug eluting stents in patients, demonstrating the need for an advanced local delivery approach for the use of metallic stents to treat vascular disease.

Methods and devices have been proposed for delivery of magnetizable therapeutic agent or agent-containing magnetic carrier to specific locations in the body. See, for example, Chen, U.S. Pat. No. 5,921,244, the disclosure of which is incorporated herein by reference. However, these magnetically susceptible therapeutic agents must be administered in the vicinity of the treatment site.

Thus, a need exists for a therapeutic system that uses stents, and/or other implantable devices, for local delivery of a therapeutic agent that would allow for the repeated administration of the same or different therapeutic agent, and, further, would have the option of locally injecting, or alternatively, peripherally administering, the therapeutic agent.

### SUMMARY OF THE INVENTION

In one aspect, the invention is a therapeutic system that uses stents, and/or other implantable devices, for local delivery of a therapeutic agent. In another aspect, the invention is a method for using stents, and/or other implantable devices, for local delivery of a therapeutic agent. The method allows for the repeated re-administration of the same or different therapeutic agent, and, further, has the option of locally injecting, or alternatively, peripherally administering, the therapeutic agent. The therapeutic system and method can be used in the treatment of chronic diseases, such as, for example, arteriosclerosis.

In one aspect, the invention comprises a magnetically assisted therapeutic system comprising:

(a) a therapeutic formulation administered to a mammalian subject by peripheral intravenous administration, in which the therapeutic formulation comprises particles, such as nanoparticles, of a magnetic or magnetizable material that carry a therapeutic agent;

(b) an implantable device implanted in a vascular system of a mammalian subject, the implanted implantable device comprising a biocompatible magnetic or magnetizable material; and

(c) a retrieval system having a magnetic or magnetizable mesh operably connected to the mammalian subject.

In one aspect of the invention, the implantable device is a stent.

In another aspect, the invention is a method for administering a therapeutic agent that comprises the steps of:

(a) intravenously administering a therapeutic formulation to a vascular system of a mammalian subject, in which the therapeutic formulation comprises particles of a biocompatible magnetic or magnetizable material that carry the therapeutic agent;

(b) delivering a portion of the therapeutic formulation to the proximity of an implantable device implanted in the vascular system in the mammalian subject by externally generating a magnetic field gradient on the implantable device, in which the implantable device comprises a biocompatible magnetic or magnetizable material; and

(c) removing a portion of the therapeutic formulation that is not delivered to the proximity of the implantable device from the vascular system.

The magnetic or magnetizable particles that carry the therapeutic agent are sequestered in the proximity of the implanted device. Particles that do not localize on the implanted device are retrieved by the mesh to prevent them from accumulating in a reticulo-endothelial system of the mammalian subject. A directable magnetic field gradient is also provided for directing the magnetic or magnetizable carrier in proximity to the implanted device.

For therapeutic treatment, the steps can be repeated, in order, as often and as frequently as required to provide the desired level of treatment.

### BRIEF DESCRIPTION OF THE DRAWINGS

The invention will be described in conjunction with the following drawings in which like reference numerals designate like elements and in which:

FIG. 1 is a block diagram illustrating an exemplary magnetically assisted therapeutic system according to an embodiment of the invention;

FIG. 2 is a flowchart illustrating an exemplary method for administering a therapeutic agent to an implanted device and for retrieving magnetic carrier nanoparticles that do not localize on the implanted device, according to an embodiment of the invention;

FIG. 3A summarizes an exemplary embodiment of the magnetically assisted therapeutic system, in which albumin modified magnetic carrier nanoparticles with a red fluorescent label were injected Into a rat having an intravascularly implanted steel stent;

FIG. 3B summarizes results of the therapeutic agent delivery, for sequestering in the implanted device;

FIG. 4 summarizes schematically the retrieval system shown in FIG. 1 that is used to model the retrieval of magnetic carrier nanoparticles or cells from the cardiovascular circulation cycle;

FIG. 5 summarizes exponential depletion kinetics of carrier nanoparticles over time under the influence of a magnetic field gradient;

FIG. 6 summarizes exponential depletion kinetics of carrier cells over time under the influence of a magnetic field gradient;

FIG. 7 summarizes how different magnetic sequestering configurations, for performing the exemplary method shown in FIG. 2, affect depletion kinetics;

FIGS. 8A and 8B summarize results of transmission electron microscopy and magnetic moment versus magnetic field (magnetization curve) for Albumin-stabilized superparamagnetic nanoparticles (MNP);

FIG. 9A-9C summarize in vitro MNP cell loading studies with respect to the kinetics of MNP uptake, cell viability and a magnetization curve of cells loaded with MNP;

FIGS. 10A-10C summarize results of magnetic cell capture under flow conditions of in vitro and in vivo;

FIGS. 11A and 11B summarize results of using bovine aortic endothelial cells (BAEC) cells co-treated with MNPs and luciferase encoding adenovirus to determine cell localization to implanted stents in vivo under interrupted flow conditions; and

FIGS. 11C and 11D summarize results of using BAEC cells co-treated with MNPs and luciferase encoding adenovirus to determine cell localization to implanted stents in vivo under uninterrupted flow conditions.

### DETAILED DESCRIPTION OF THE INVENTION

The invention provides magnetic gradient targeting, sequestering and retrieval of magnetic or magnetizable therapeutic formulations and magnetically assisted or induced therapeutic systems manufactured therefrom. This is achieved using peripheral intravenous administration of a magnetic or magnetizable therapeutic formulation without requiring localized invasive delivery at the site of the implantable device. The therapeutic formulation comprises particles of a biocompatible magnetic or magnetizable material that carry a therapeutic agent.

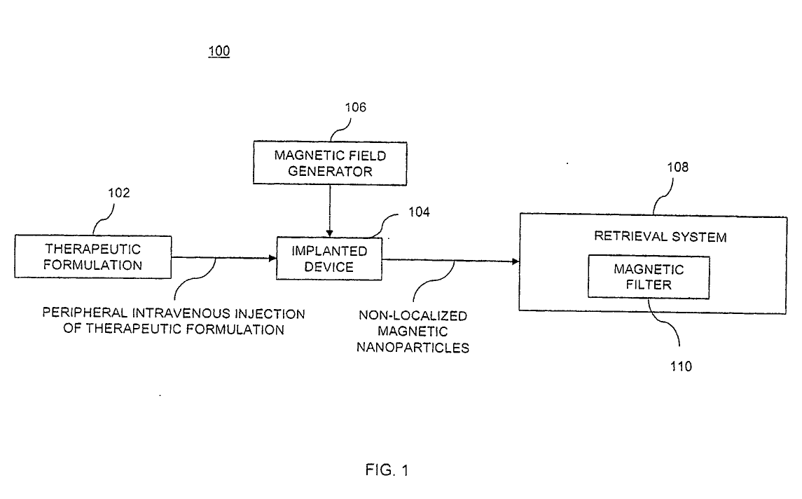

Referring to FIG. 1, an exemplary magnetically assisted therapeutic system 100 is illustrated. In practice, therapeutic system 100 typically comprises an implanted implantable device 104 that has been implanted in a mammalian subject (not shown), magnetic field generator 106, such as a magnet, that externally generates a magnetic field gradient on the implanted device 104 and a magnetic or magnetizable therapeutic formulation 102 that has been administered to the subject by peripheral intravenous administration. Device 104 is typically a vascular implantable device that has been implanted in the vascular system of the mammalian subject. The therapeutic formulation 102 may be administered through a vein, in for example, an appendage. The particles of the therapeutic formulation 102 can be surface modified to extend the intravascular circulatory time, thereby permitting adequate to optimal numbers of cardiac cycles for optimized implanted device uptake.

Particles that are not sequestered in proximity to the implanted device 104 are removed from circulation by a retrieval system 108 so that they do not accumulate in the reticulo-endothelial system, where they might have undesirable side effects. In addition, wide biodistribution of the magnetic or magnetizable carriers included as a part of the therapeutic formulation is also minimized. The retrieval system 108 makes use of apheresis principles but provides a magnetic mesh filter 110 placed in the circulation circuit.

### Implantable Device

The implantable device 104 comprises a biocompatible magnetic or magnetizable material. The device is typically implanted in the vascular system of a mammalian subject. The device must be biocompatible and must comprise a material that is either magnetic, or magnetizable (i.e., capable of being magnetized). Stainless steel, for example, Grade 304 Stainless Steel, a widely used stainless steel, can be used in the implantable device 104.

Provided they comprise a material that is biocompatible and is either magnetic, or magnetizable, implantable devices appropriate for the delivery system include, but are not limited to, stents, heart valves, wire sutures, temporary joint replacements and urinary dilators. Other suitable medical devices for this invention include orthopedic implants such as joint prostheses, screws, nails, nuts, bolts, plates, rods, pins, wires, inserters, osteoports, halo systems and other orthopedic devices used for stabilization or fixation of spinal and long bone fractures or disarticulations. Other devices may include non-orthopedic devices, temporary placements and permanent implants, such as traceostomy devices, jejunostomy and gastrostomy tubes, intraurethral and other genitourinary implants, stylets, dilators, stents, vascular clips and filters, pacemakers, wire guides and access ports of subcutaneously implanted vascular catheters. A preferred implantable device is a stent. Surface modification of metal supports to improve biocompatibility is disclosed in Levy, U.S. Patent Publication 2003/0044408, the disclosure of which is incorporated herein by reference.

### Therapeutic Formulation

The therapeutic formulation 102 comprises particles of a biocompatible magnetic or magnetizable material that carry a therapeutic agent or comprise magnetically-responsive cells. Magnetic nanoparticles include particles that are permanently magnetic and those that are magnetizable upon exposure to an external magnetic field but lose their magnetization when the field is removed (superparamagnetic). Superparamagnetic particles are preferred to prevent irreversible aggregation of the particles. A therapeutic agent includes any material that is desired to be administered to a mammalian subject using the system and method of the invention.

### Therapeutic Agent

Suitable therapeutic agents include, for example, pharmaceuticals, nucleic acids, such as transposons, signaling proteins that facilitate wound healing, such as TGF-β, FGF, PDGF, IGF and Gh proteins that regulate cell survival and apoptosis, such as Bcl-1 family members and caspases; tumor suppressor proteins, such as the retinoblastoma, p53, PAC, DCC.Nfl, NF2, RET, VHL and WT-1 gene products; viral vector systems; extracellular matrix proteins, such as laminins, fibronectins and integrins; cell adhesion molecules such as cadherins, N-CAMS, selectins and immunoglobulins; anti-inflammatory proteins such as Thymosin beta-4, IL-10 and IL-12. Examples of viral vector systems include adenovirus, retrovirus, adeno-associated virus and herpes simplex virus. Suitable therapeutic agents within these classes and other suitable therapeutic agents that can be used in the practice of the invention will be apparent to those skilled in the art. Typically, the therapeutic agent selected will be administered to a mammalian subject, such as human, in need of the treatment provided by the therapeutic agent.

### Particles

The therapeutic formulation comprises nanoparticles with a permanently magnetic or a magnetizable (superparamagnetic) material in their composition. Mixed iron oxide (magnetite), as well as substituted magnetites that include additional elements (e.g. zinc), in the form of small sized nanocrystals retaining no magnetization upon magnetic field removal are an example of superparamagnetic materials useful for biomedical applications. The magnetic responsiveness of individual superparamagnetic nanocrystals typically sized below 20 nm is, however, too small to allow for efficient control of their biodistribution using magnetic forces.

One approach to overcome this limitation, while retaining superparamagnetism essential for the safe use of the nanoparticles, is to incorporate a large number of individual magnetite nanocrystals in a larger sized composite made of a water-insoluble biocompatible material, usually a polymer, which may be either biodegradable or non-biodegradable. Examples of such polymeric materials are poly(urethane), poly(ester), poly(lactic acid), poly(glycolic acid), poly(lactide-co-glycolide), poly(ε-caprolactone), poly(ethyleneimine), poly(styrene), poly(amide), rubber, silicone rubber, poly(acrylonitrile), poly(acrylate), poly(metacrylate), poly(α-hydroxy acid), poly(dioxanone), poly(orthoester), poly(ether-ester), poly(lactone), mixtures thereof and copolymers of corresponding monomers.

Such polymeric nanoparticles with incorporated superparamagnetic nanocrystals may be prepared, for example, by dispersing the superparamagnetic nanocrystals in an organic solvent, in which the polymer and/or the therapeutic agent is dissolved, emulsifying the organic phase in water in the presence of a suitable stabilizer, and finally eliminating the solvent to obtain solidified nanoparticles. Conditions of nanoparticle preparation should not be damaging for the therapeutic agent to be attached. For example the temperature is typically about 25° C. to about 37° C. Alternatively, or additionally, the therapeutic agent may be attached, or “tethered”, to the surface of pre-formed nanoparticles either by adsorption, charge complexation, or covalent binding. The magnetic nanoparticles that carry the therapeutic agent typically have an average diameter of about 50 nm to about 500 nm, for example about 200 nm to about 400 nm.

Preparation of Supermagnetic Nanoparticles for Biological Applications is Described in, for example, Cui, U.S. Pat. No. 7,175,912, the disclosure of which is incorporated herein by reference; Hu, U.S. Pat. No. 7,175,909, the disclosure of which is incorporated herein by reference; and Gruettner, U.S. Patent Publication 2005/0271745, the disclosure of which is incorporated herein by reference. Magnetic nanoparticles, information for the development of magnetic nano-particles, and regents for the preparation of magnetic nanoparticles (MNP) are available from Ferrotec Corporation, Bedford, N.H., USA.

Various procedures for associating therapeutic agents with magnetic nanoparticles so that the therapeutic agent is carried by the nanoparticle have been described in, for example, Chen, U.S. Pat. No. 7,081,489, the disclosure of which is incorporated herein by reference; Kresse, U.S. Pat. Nos. 6,048,515, and 6,576,221, the disclosures of which are incorporated herein by reference; and Bahr, U.S. Pat. No. 6,767,635, the disclosure of which is incorporated herein by reference.

The surface of the particle may be modified to allow for its chemical derivatization with a biomaterial. In one procedure, the particles can be coated with a thiol-reactive and photoactivatable polymer. Irradiation results in covalent binding of the polymer to the surface, and its thiol-reactive groups can subsequently be used to attach agents providing stealth properties in the blood circulation (see below), and/or specific binding to a target tissue. Photochemical activation of surfaces for attaching biomaterial is disclosed in Alferiev, U.S. Patent Publication 2006/0147413, the disclosure of which is incorporated herein by reference.

Extended circulation time of the magnetic nanoparticles that carry the therapeutic agent (i.e., “modified magnetic nanoparticles”) can be achieved by preventing their rapid opsonization and subsequent clearance by reticulo-endothelial system by doing one of the following: they can be coated with a biocompatible hydrophilic polymer (e.g., polyethyleneglycol, dextran), or, alternatively, surface modified with serum albumin that prevents or delays binding of opsonins to their surface. Procedures for preparing these polymers are given in the Examples. As described in the Examples, magnetic nanoparticles that carry D1, IgG and adenovirus have been prepared. Adenovirus is a promising gene vector for therapeutic applications. It should be understood that these embodiments are non-limiting examples.

### Method

### Administration of the Therapeutic Formulation

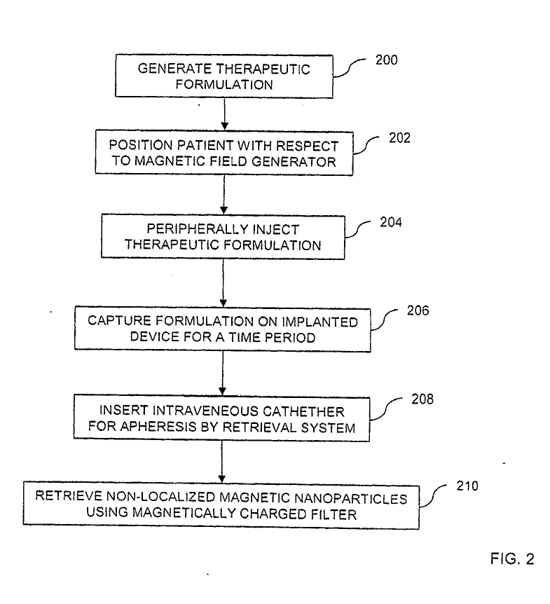

Referring now to FIG. 2, an exemplary method is described for administering a therapeutic agent to an implanted device and for retrieving magnetic carrier nanoparticles that do not localize on the implanted device, such as for clinical use. In step 200, a therapeutic formulation is generated according to the needs of the patient that includes an implanted device. For example, the patient may include a need for primary drug administration, a change in a drug, a change in a dose, multiple drug administration, gene therapy, or cell therapy. In step 202, the patient is positioned with an external magnet over the site of the implantable device (such as a stent) deployment.

In step 204, the therapeutic formulation is peripherally intravenously injected. For example, the therapeutic formulation may be injected in an arm vein where the therapeutic formulation is formed of a suspension of magnetic nanoparticles containing the therapeutic agent of interest. As another example, the injection may also consist of stem cells loaded with magnetic nanoparticles. Although the injection is described as being peripherally intravenously injected, it is contemplated that the injection may be performed at the site of the implanted device. The amount of the therapeutic formulation injected vary depending on the purpose of delivery, e.g., prophylactic, diagnostic, therapeutic, etc. and on the nature of the therapeutic agent involved. This amount can be determined by those skilled in the art.

In step 206, following the injection, capture of the therapeutic formulation by the implanted device is provided for a period of time. Although in an exemplary embodiment this duration may be in the range of about 15-30 minutes, it is understood that any suitable duration for capture of therapeutic formulation by the implanted device may be used. As described herein, the nanoparticle surface may be chemically modified to avoid rapid clearance by the reticulo-endothelial system.

In step 208, following the intravenous injection and magnetic localization, the patient undergoes a second intravenous catheter placement for apheresis, for example, by the retrieval system 108 (FIG. 1). In this manner, there are two catheter lines to cycle through the retrieval system 108 (FIG. 1). In step 208, non-localized magnetic nanoparticles are retrieved using the magnetic filter 110 (FIG. 1) via an apheresis process that allows enough passages to remove substantially all of the non-localized magnetic nanoparticles.

Sequester refers to a magnetically induced sequestering of the particles of the therapeutic formulation as a result of a magnetic field gradient generated externally on an implanted intravascular device in a mammalian subject. Sequestering is also referred to as magnetically assisted “trapping” or “filtering.” The terms “retrieve” or “retrieval” refer to a magnetically induced and directed movement or sequestering of the particles of the therapeutic formulation as a result of applying a magnetic field gradient generated externally on the mammalian subject.

According to an exemplary embodiment, the invention provides peripheral intravenous magnetic nanoparticle administering with localization in an arterial stent in a mammalian subject (e.g. rat as the mammalian subject model).

### Retrieval of the Un-Sequestered Therapeutic Formulation

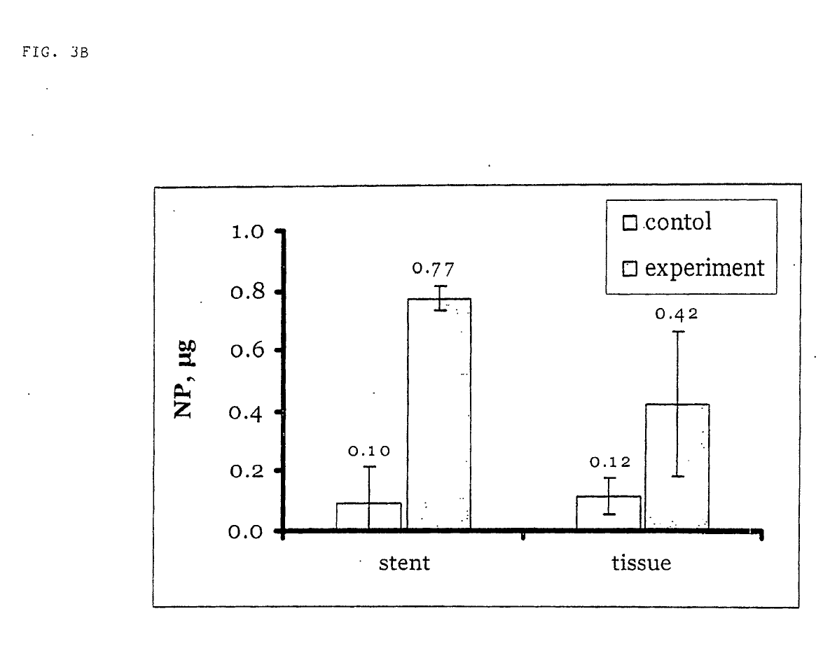

Magnetic separation using a peripheral mesh operably connected to the mammalian subject is used in the filtering system as part of the therapeutic system that is inserted into an apheresis apparatus. Magnetic separation removes the particles that have not been sequestered (i.e., localized on the implanted device) to prevent them from accumulating in a reticulo-endothelial system of the mammalian subject. As shown in FIG. 4, flow system 400 includes a magnetic trap 402, electromagnets 404 for generating a magnetic field, a peristaltic pump 406, a stirrer 408, and faucets 410 for directing flow to cycle A or cycle B. Operation of this system is described in Example 2.

### INDUSTRIAL APPLICABILITY

The invention provides therapeutic formulations and systems that deliver-therapeutic agents to a specific site of treatment and removes therapeutic agents not delivered to the site of treatment. The therapeutic formulation and system are used in combination with surface modification of inert surfaces useful for implantation, which permits attachment of molecular therapeutics such as proteins, genes, vectors, or cells and avoid using organic solvents that can potentially damage both the surface and molecular therapeutics.

Use of peripheral intravenous administration of magnetic nanoparticles, followed by magnetic targeting to stents and/or other implantable devices, followed by retrieval of un-sequestered particles, can be used to treat virtually any disorder that can be accessed through vascular means, or any disorder for which intravascular therapy is optimal compared to gastrointestinal administration. It more effectively treats arterial disease (with additional courses of various therapies) in a patient that has already been subjected to metallic stent angioplasty. For example, pulmonary hypertension is now treated with peripheral intravenous administration of vasodilators, often using drug pumps. This approach is minimally effective and has serious side effects. In patients with pulmonary hypertension, it is contemplated that stents are deployed in the main or branch pulmonary arteries, and magnetic nanoparticles containing potent pulmonary vasodilator agents are then be injected and localized on to these stent structures thus providing local delivery to the pulmonary vasculature and optimizing the therapy for this difficult disorder. In addition, virtually any intravascular metallic implant (e.g., nonvascular, such as a bronchial stent) could also be adapted to take advantage of this approach.

The invention can be used in cell delivery experiments, in view of magnetic-stent mesh targeting results shown, to address two cell delivery major issues. First, the results demonstrate that cells can be targeted to a stent by a magnetic field gradient generated on the stent by a uniform magnetic field, and thus, this approach will likely be comparably successful in-vivo. Secondly, these data also demonstrate that the same magnetic trapping principles used to remove excess non-targeted particles can also be used to retrieve and remove cells that are not localized to a desired site.

Cell therapy at this time is just beginning early stages of clinical investigations, with mixed to poor results. One of the great problems with all of the cell therapy strategies is use thus far for either heart failure, tissue engineering, cell seeding of implants etc., is a failure to properly target and retain cells at the desired site. This has been most apparent in the cell therapy studies for heart failure thus far, where more than 95% of cells injected directly into the myocardium are lost due to circulatory clearance. The magnetic gradient targeting of cells loaded with magnetic nanoparticles offers one potential solution to the problem.

The advantageous properties of this invention can be observed by reference to the following examples, which illustrate but do not limit the invention.

### EXAMPLES

### Procedure for Preparation of Surface Modified Particles

Extended circulation time of the magnetic nanoparticles that carry the therapeutic agent (“modified magnetic nanoparticles”) can be achieved by coating with a biocompatible hydrophilic polymer or, alternatively, surface modification with serum albumin. Preparation of either type of modified particles includes a common step of producing a magnetically responsive agent, iron oxide. Fine dispersion of iron oxide in a suitable organic solvent is typically obtained as follows: an aqueous solution containing ferric and ferrous chlorides is mixed with an aqueous solution of sodium hydroxide. The precipitate is coated with oleic acid by short incubation at 90° C. in ethanol. The precipitate is washed once with ethanol to remove free acid and dispersed in chloroform.

The resulting organic dispersion of iron oxide in chloroform is used to dissolve a biodegradable polymer, polylactic acid (PLA) or its polyethyleneglycol conjugate (PLA-PEG), thus forming an organic phase. The organic phase is emulsified in an aqueous albumin solution (1%) by sonication on an ice bath followed by evaporation of the organic solvent. The particles are separated from the unbound albumin by repeated magnetic sedimentation/resuspension cycles.

Alternatively, a post-formation surface modification can be used. In this case, particles are formed as described above using a photoreactive polymer (a PBPC/PBMC (polyallylamine-benzophenone-pyridyldithio/maleimido-carboxylate polymer) as a stabilizer in the aqueous phase. Subsequent brief ultraviolet irradiation achieves covalent binding of the polymer to the magnetic nanoparticle. The resulting particles are reacted in suspension with a thiolated polyethyleneglycol, which allows better control over the particle size and the extent of surface modification. However, this procedure may not be suitable for use with photochemically labile therapeutic agents.

Albumin-coated and PLA-PEG magnetic particles typically have an average size of 200-260 nm. Particles surface-modified with polyethyleneglycol post-formation are typically 300-380 nm. All these particles exhibit superparamagnetic properties (i.e. have no magnetic remnants, which is critical in order to prevent potentially hazardous irreversible aggregation triggered by magnetic field exposure) and strong magnetic responsiveness as compared to commercially available magnetic particles that comprise non-biodegradable polymers.

### Example 1

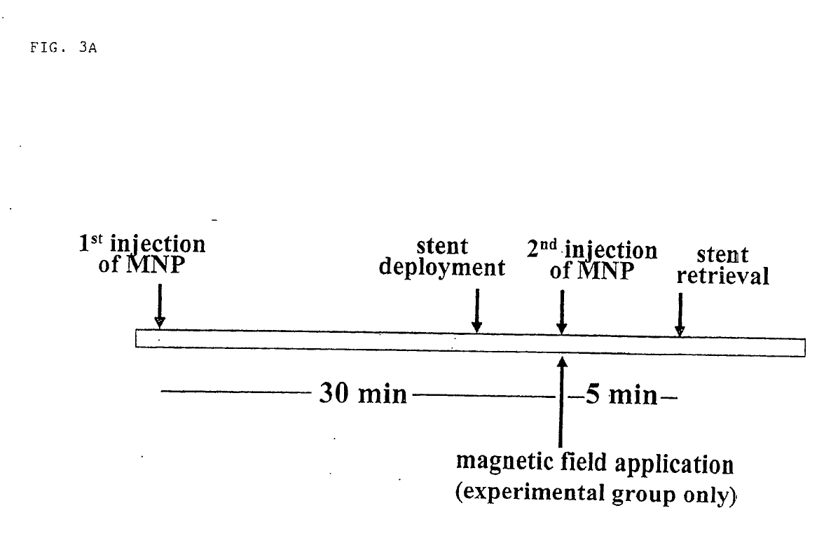

Referring now to FIGS. 3A and 3B, this example illustrates magnetic gradient targeting of nanoparticles. Albumin modified magnetic nanoparticles with a red fluorescent label were injected into the tail vein of a rat with an already deployed 6 mm-long Grade 304 Stainless Steel stent (FIG. 3A). Grade 304 Stainless Steel (“304 steel”) may potentially be approved by the FDA for use in implantable devices. Although there are no commercially available stents made out of 304 steel, a stent design was created and contracted to a medical device company to fabricate a set of these stents for use in the experiments. Thus, all of the studies reported here did not use any of the currently commercially used stents.

The 304 stent in these rat studies was investigated both with and without a magnetic field across the stent. In addition, magnetic nanoparticles without a stent were also injected into animals, with investigations to see if there was any localization that took place without stent deployment.

Methods: Paclitaxel was dispersed within the polylactic acid (PLA) matrix of magnetite-loaded nanoparticles (MNP). Adenovector-tethered MNP were prepared using photochemical surface activation with the subsequent attachment of a recombinant adenovirus binding protein, D1, and then end formation of nanoparticle-adenovirus complexes. Plasmid vectors were charge-associated with PEI-functionalized MNP. Magnetic trapping of MNP on the steel meshes and stents under different field strength and flow conditions was studied in a closed circuit flow system. Transfection/transduction using gene vectors associated with magnetic nanoparticles was studied in smooth muscle (SMC) and endothelial cells. Magnetic force-driven localization of reporter gene-associated MNP and MNP-loaded cells on pre-deployed stents and resulting transgene expression were studied a rat carotid stent model.

Protocol (FIG. 3A): Four hundred μl of magnetically responsive fluorescent labeled, polylactic acid based magnetite-loaded nanoparticles were intravenously-injected (through the tail vein) upon induction of anesthesia in 480-510 g rats (Sprague-Dawley rats (n=6)). The magnetite-loaded nanoparticles were 350 nm, consisting of 7.2 mg per injection. This injection was carried out to saturate the reticulo-endothelial system of the animal to prevent excessive capturing of the second main dose of nanoparticles in liver and spleen.

Within 30 minutes of the first injection, a 304 steel stent was deployed in the left common carotid artery. Immediately after that, another 400 μl dose of the nanoparticles was injected intravenously, either with or without 300 G magnetic field created by 2 electromagnets placed adjacent to the neck of the animal. The field was maintained for 5 min after injection, after which the arteries were harvested. The stents were removed and nanoparticles deposition on stents and luminal aspects of arteries was examined by fluorescence microscopy. After acquisition of respective images BODIPY-labeled (red fluorescent) PLA was extracted in acetonitrile and its concentration was determined fluorimetrically against a calibration curve. For fluorescence control/background purposes in one additional rat no nanoparticles were injected and the stented arteries were removed and similarly processed to obtain background fluorescence values.

Results: In a closed circuit flow system MNP and cells loaded with MNP were trapped on magnetic meshes with exponential kinetics. Rat aortic SMC (A10) cultured on 316L stainless steel grids showed 100-fold increased gene transduction when exposed to the MNP-Ad_{GFP} compared to controls. Paclitaxel MNP demonstrated inhibition of A10 cells growth in culture. Systemic intravenous injection in rats of MNP resulted in 7-fold higher localization of MNP on intra-arterial stents compared to controls when carried out in the presence of external magnetic field (300-G).

The results of these studies are shown in FIG. 3B (flourimetry, 540/575 nm), as well as with fluorescent microscopy (not shown), demonstrating intense localization of magnetic nanoparticles to the deployed 304 stent, and also localization of magnetic nanoparticles to the arterial wall directly proximal to the stent. In addition, using a specific fluorescent assay, the significant localization of magnetic nanoparticles following intravenous injection using this methodology was quantified.

Conclusion: Magnetically targeted drug/gene delivery using high field gradients to stented arteries offers great promise because of the potential for not only initial dosing, but repeated administration utilizing magnetic field-mediated localization of vectors to the stented arterial wall. These results clearly demonstrate a significantly higher nanoparticles deposition on stents and adjacent arterial tissue in the group where systemic intravenous delivery was carried out in conjunction with an electromagnetic field compared to “no field” controls. Non-stented arteries demonstrated no nanoparticle localization with or without a magnetic field.

### Example 2

This Example illustrates removal of residual nanoparticles and cells with an external magnetically responsive steel filter (“magnetic trap”). FIG. 4 illustrates a flow system 400 that schematically summarizes the retrieval system 108 (FIG. 1) that is used to model the retrieval of magnetic nanoparticles or cells from the circulation. As shown in FIG. 4, flow system 400 includes a magnetic trap 402 (an Eppendorf with 430 stainless steel mesh for capturing of the residual nanoparticles), electromagnets 404 for generating a magnetic field, a peristaltic pump 406, a stirrer 408, and faucets 410 for directing flow to cycle A or cycle B. A suitable peristaltic pump 406, stirrer 408, and faucets 410, as commonly for an apheresis apparatus, will be understood by the skilled person from the description herein.

The following experimental protocol was used to determine the kinetics of magnetic nanoparticles and cell capture, respectively, using the “Magnetic Trap” apparatus.

PLA-PEG based magnetic nanoparticles were diluted in 50 ml of 5% glucose solution and filtered (5 μm cut-off) to ensure uniform particle size. Alternatively, bovine aortic endothelial cells (BAECs) were grown to confluence and incubated with fluorescently labeled magnetic nanoparticles on a cell culture magnet (Dexter Magnet Technologies, Elk Grove Village, Ill.) producing a strong magnetic field (500 Gauss) for 24 hours, followed by cell washing and resuspension in fresh cell culture medium. Untreated cells were used as a control.

The flow system 400 was purged with 5% glucose or cell culture medium, respectively, (washing step) followed by one cycle of nanoparticle/cell suspension in the loop A to equilibrate the system (priming step). Next, nanoparticle/cell suspension was redirected to the loop B including the trapping device 402 equipped with one or three 430 stainless steel mesh pieces (total weight of 0.30±0.01 and 0.83±0.05 g, respectively) and an external magnetic field of 800 Gauss generated by two solenoid electromagnets 404. A to sample was withdrawn and further used as a reference (100% of NP/cells). Additional samples were collected at predetermined time points during 2.5 hours and 35 min in the nanoparticles and cell retrieval experiments, respectively. The effect of the magnetic field exposure was Investigated In comparison to “no field” conditions employed during the first 25 and at 3 minutes into the experiment for the nanoparticles and cells, respectively, after which the field was applied. A NP/cell fraction remaining in the circulation at a given time point was determined fluorimetrically (λₑₓ=540 nm, λₑₘ=575 nm) in relation to the reference sample. The mesh samples were visualized under the fluorescent microscope using red fluorescence filter set (540/575 nm) immediately and 24 hours after completing the experiment. Collected cells were incubated overnight at 37 C and their morphology was examined microscopically.

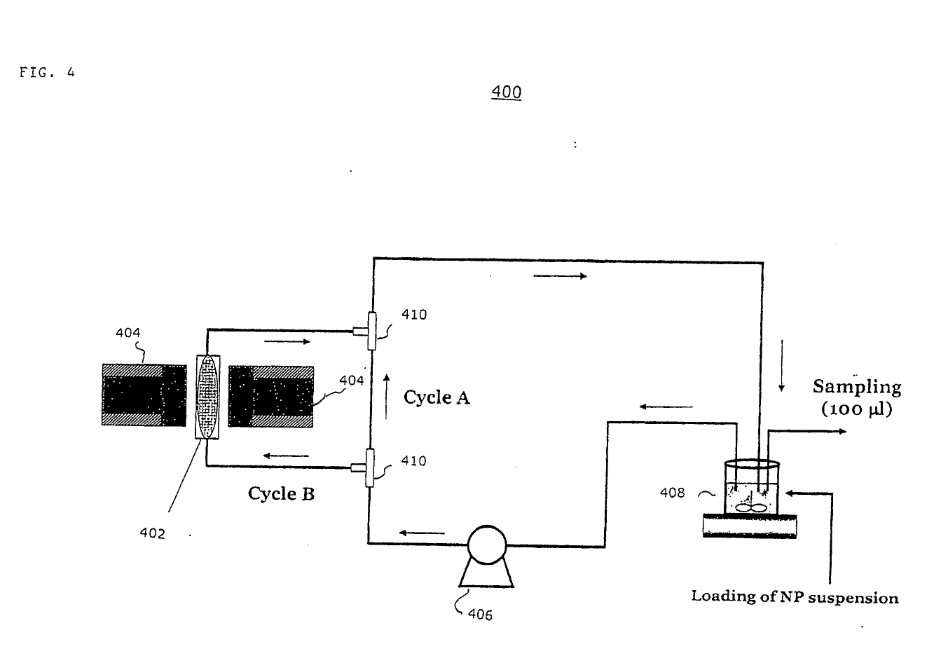

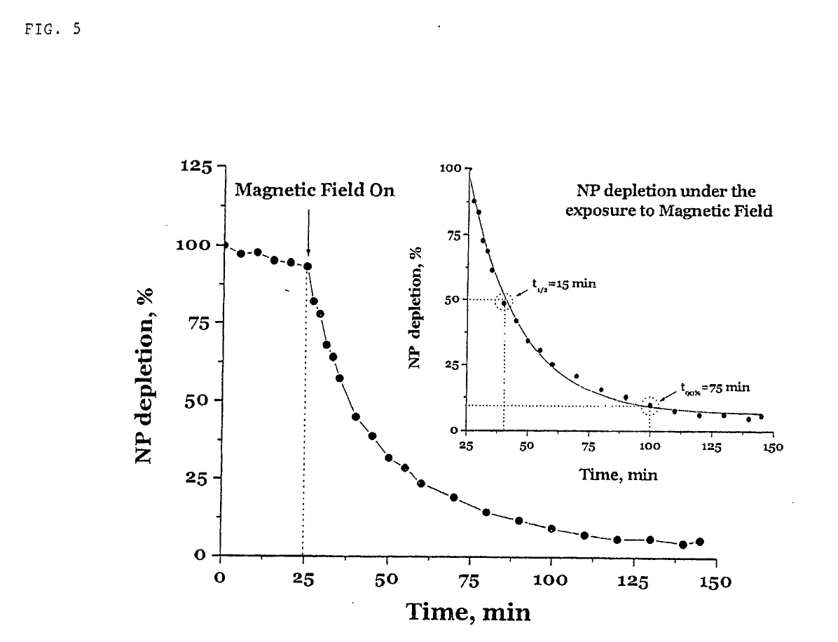

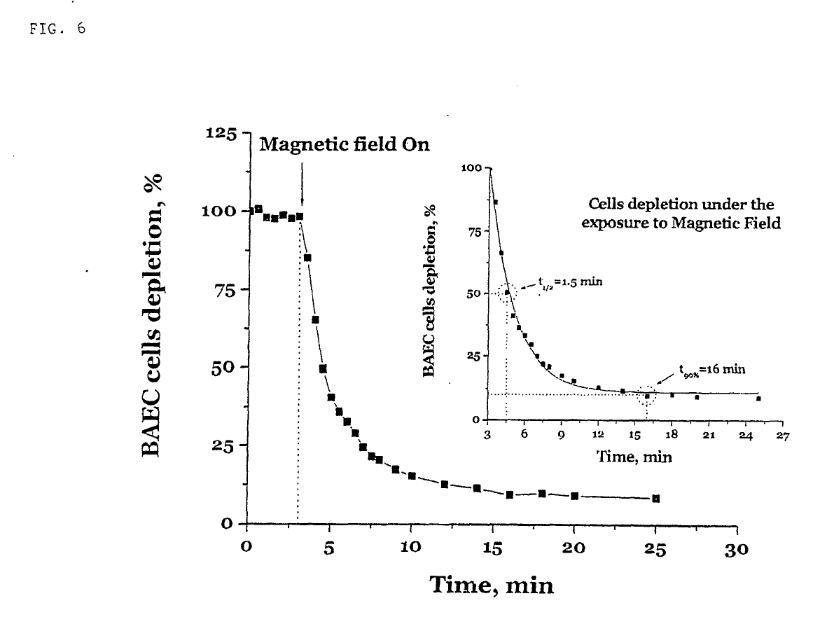

FIG. 5 and FIG. 6 depict exponential depletion kinetics of nanoparticles and BAEC cells, respectively, over time under the influence of a magnetic field. A significantly less pronounced decrease in both nanoparticles and BAEC cells is also observed in “no field” conditions. Under the magnetic field exposure, the depletion kinetics of both nanoparticles and cells was very fast with t_{90%} (i.e., time required to eliminate 90% of the circulating nanoparticles or cells) equaling 75 min and 16 min for nanoparticles and cells, respectively. The five-fold lower t_{90%} for cell capture is apparently due to their higher magnetic responsiveness due to the cells containing a large number of nanoparticles/cell compared to that of the smaller sized NP.

Referring to FIG. 7, different magnetic trap configurations and corresponding depletion kinetics are shown. Increasing the amount and surface area of the 430 stainless steel in the “Magnetic Trap” from 0.3 to 0.83 g, caused a significant decrease in the circulation t_{1/2} of the nanoparticles (27 vs. 50 min). Thus, optimization of the “Magnetic Trap” design could potentially allow for nanoparticles and cell retrieval kinetics sufficiently fast for its clinical use. Spreading of cells was also demonstrated where the cells were removed from the circulation for measurement of cell depletion. Cells were grown overnight on the cell culture plate at the 37° C. and in the atmosphere of 5% of CO₂. Micrographs of the mesh taken post experiment demonstrated nanoparticles deposited on the “Magnetic Trap.”

Magnetically responsive cells captured at the end of the experiment and spreading of the cells 24 hours later were also demonstrated. Cells sampled from the circulation during the cell capture experiment demonstrate normal morphology characteristic of BAEC. The growth conditions are 10% FBS supplemented DMEM at 37° C. and 5% CO₂. The meshes used in the magnetic trap in this experiment were visualized under the fluorescent microscope immediately and 24 hours post experiment in order to evaluate the morphology of the captured cells. A high number of cells are shown to be initially captured by the edges of the mesh, of which those located most adjacent to the mesh surface form a layer of uniformly spread cells after 24 hours over the expanse of the entire surface of the mesh framework thus showing the viability of the magnetically targeted cells. Capture of magnetic carrier nanoparticles at the end of experiment was demonstrated on the surface of the 430 stainless steel mesh under the field of 800 Gauss (“The Magnetic Trap”), as compared with a control mesh at the beginning of the experiment before application of magnetic field.

### Example 3

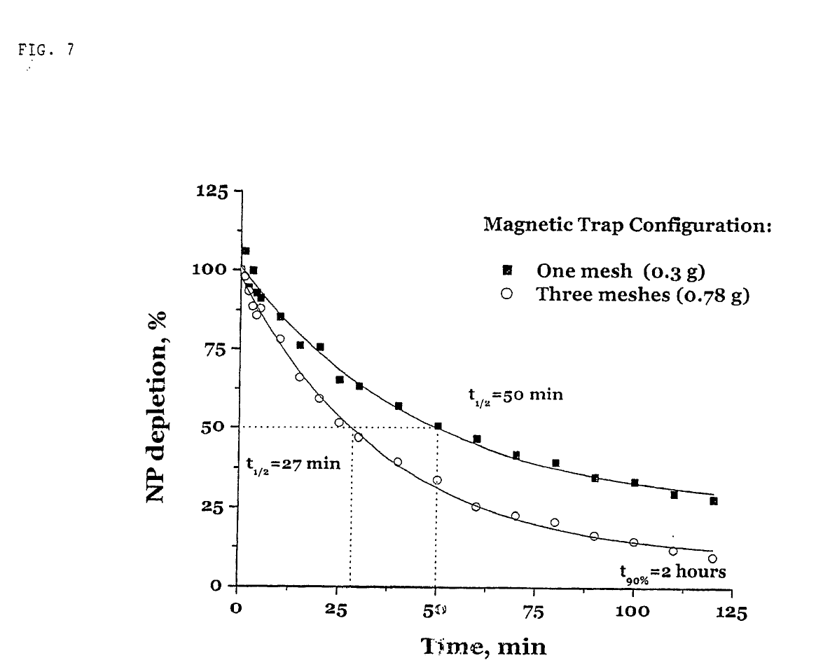

Referring now to FIGS. 8A and 8B, results from transmission electron microscopy and a magnetization curve (magnetic moment versus magnetic field) are shown, respectively for Albumin-stabilized magnetic nanoparticles (MNP), described above with respect to Example 1. Note the small size and the large number of individual oleic acid coated magnetite grains distributed in the MNP polymeric matrix (FIG. 8A). MNP exhibits a superparamagnetic behavior, showing no significant hysteresis, and a remnant magnetization on the order of 0.5% of the respective saturation magnetization value (FIG. 8B).

### Example 4

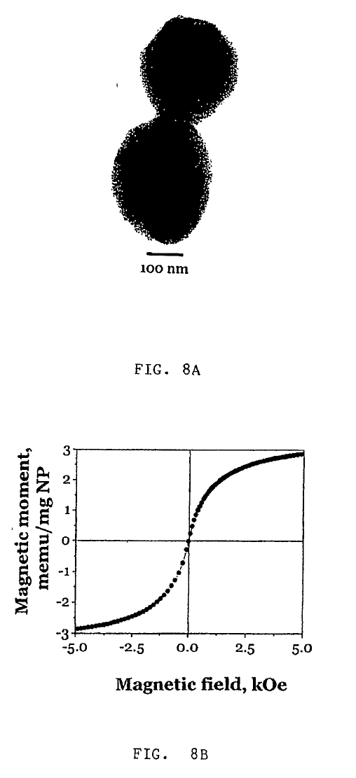

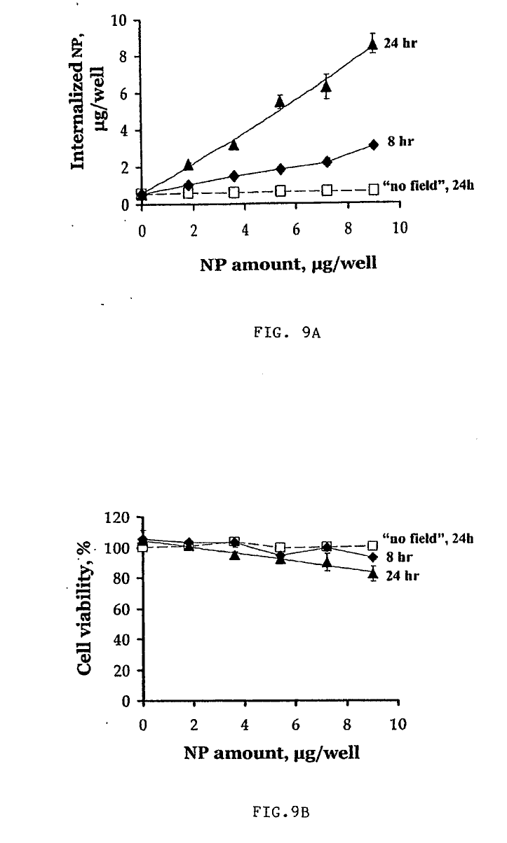

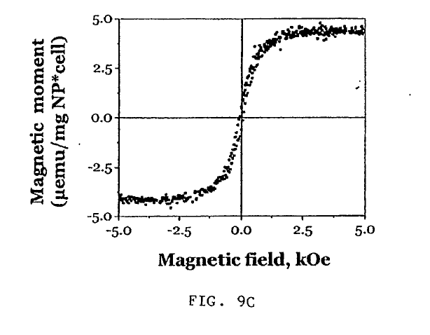

Referring now to FIGS. 9A-9C, in vitro MNP cell loading studies are illustrated. In particular, FIG. 9A illustrates kinetics of the MNP uptake by bovine aortic endothelial cells (BAEC) as a function of MNP dose and incubation time; FIG. 9B illustrates cell viability as a function of MNP dose and incubation time; and FIG. 9C illustrates a magnetization curve of cells loaded with MNP demonstrating superparamagnetic behavior as was observed with MNPs per se. The nanoparticles uptake was determined by fluorescence of internalized MNPs. Cell survival was determined by Alamar Blue assay.

BAEC (bovine aortic endothelial cells) were incubated with various doses of MNPs on a magnet. As shown in FIG. 9A, the MNP uptake was determined at different time points by fluorescence of internalized nanoparticles. The amount of internalized MNPs was near linearly dependent on the nanoparticle dose. Approximately 30% of internalization was observed after 8 hours and the uptake was practically complete after 24 hours, whereas no significant uptake was achieved in the absence of a magnetic field at 24 hr. As shown in FIG. 9B, cell viability at different experimental conditions (incubation time and MNP dose) was not adversely affected by MNP loading. Greater than 85% of cell survival was observed at all studied MNP doses and incubation times relatively to untreated cells. As shown in FIG. 9C, the magnetization curve of cells loaded with MNPs demonstrating super-paramagnetic behavior showing no significant hysteresis and a remnant magnetization on the order of 0.5% of the respective saturation magnetization value.

### Example 5

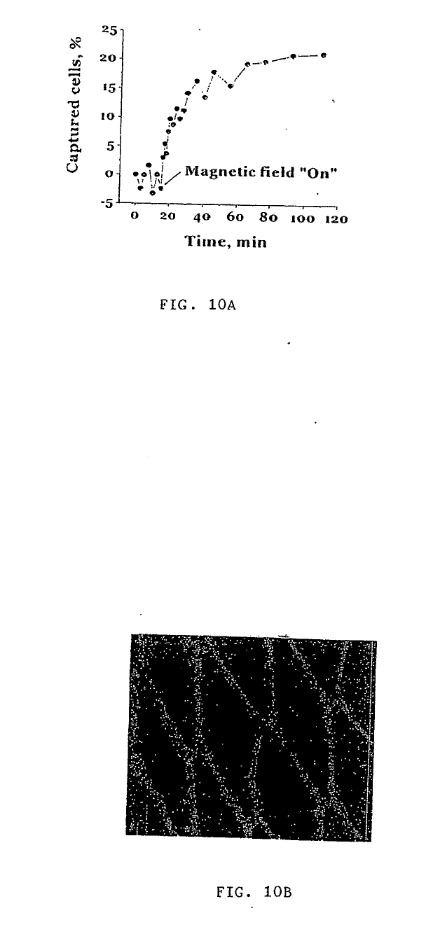

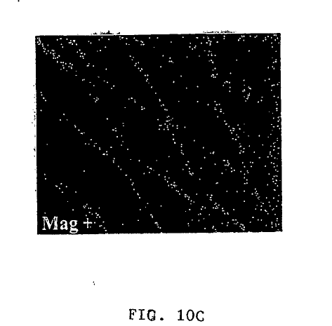

Referring now to FIGS. 10A-10C, magnetic cell capture on a stent is illustrated under flow conditions in vitro and in vivo. In particular, FIG. 10A illustrates in vitro capture kinetics of magnetically responsive cells (BAEC) on a 304 grade stainless steel stent under the field of 800 Gauss and flow rate of 30 ml/min, and the data is obtained by measurement of MNP fluorescence; FIG. 10B illustrates BAEC cells captured in vitro on a 304 stent highlighted by red fluorescence of MNP; and FIG. 10C illustrates BAEC cells captured in vivo on a deployed 304 stent in rat carotid artery. With respect to FIG. 10C, BAEC cells preloaded with fluorescent MNP were transthoracically injected into the left ventricular cavity. Animals were exposed to a magnetic field of 1000 Gauss during 5 min including the injection time. The animals were sacrificed 5 min after delivery, and the explanted stents were examined by fluorescence microscopy.

The behavior of magnetic cell capture on a 304 stainless steel stent in vitro was characterized using closed-loop flow system 400 (FIG. 4). BAEC cells laden with MNP circulated at a flow rate of 30 ml/min and the magnetic field of 1000 Gauss was applied. Cell depletion was monitored by measurement of MNP fluorescence and the results presented as a percent of captured cells. In the absence of a magnetic field practically no cell capture was observed. However, as shown in FIG. 10A, when a magnetic field was applied, cells displayed exponential capture kinetics with the initial rate of 1% of captured cells per min. About 50% of cells were captured on a stent within first 10 min. Qualitative result of this experiment, shown in FIG. 10B, illustrate where the cells captured on a stent are highlighted by MNP red fluorescence.

A comparable result was observed in a proof-of-concept in vivo animal experiment, employing well characterized rat carotid stenting model. Stainless steel 304 stent was deployed in the rat carotid artery. BAEC cells preloaded with fluorescent MNP were transthoracically injected into the left ventricular cavity. Animals were exposed to a magnetic field during 5 min including the injection time. Control rats underwent an identical procedure, where no magnetic field was employed. The animals were sacrificed 5 min after delivery, and the explanted stents were examined by fluorescence microscopy. As shown in FIG. 10C, the qualitative results as compared to the control rats (qualitative results not shown for the control rats) illustrate that only the presence of a magnetic field led to a cell capture on the stent.

Conclusion: Homogeneous magnetic field used in the described above rat model allowed generation of sufficient magnetic field gradients on 304 stent struts for successful capture of magnetically responsive cells from blood circulation.

### Example 6

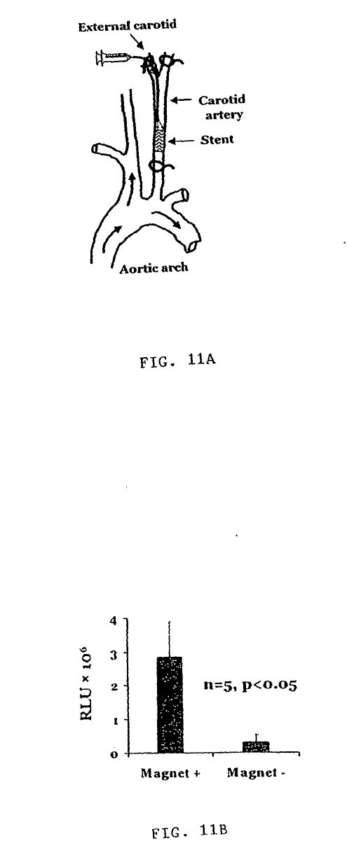

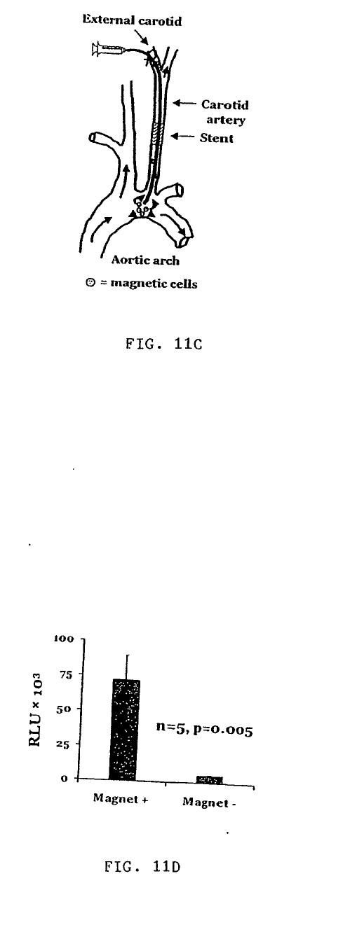

Referring now to FIGS. 11A-11D, an example illustrating in vivo local cell delivery is described. In particular, FIGS. 11A and 11B illustrate conditions under interrupted flow; and FIGS. 11C and 11D illustrate conditions under uninterrupted flow using rat carotid stent-angioplasty model.

Protocol: In order to attain greater insights regarding long term residence and functional competence of delivered cells a series of experiments were carried out using BAEC cells co-treated with MNPs and luciferase encoding adenovirus. BAEC cells were co-treated with MNP and luciferase adenovirus. Luciferase adenoviral transduction was used to determine cell localization to implanted stents in vivo by a bioluminescence technique. After adenovirus infection and preloading with MNPs the cells were locally delivered to an isolated stented segment of the rat carotid in the presence of a magnetic field (Mag+group).

Under interrupted flow (FIGS. 11A and 11B), the delivery time was extremely short, 15 seconds. The cells were then evacuated, and the magnetic field was maintained for additional 5 minutes (Mag+group).

Under uninterrupted flow (FIGS. 11C and 11D), the cells were injected during 1 min through a catheter positioned in the aortic arch and delivered to the stented carotid segment. The duration of magnetic field exposure was a total of 5 min Including injection time (Mag+group). The control rats in both experiments underwent an identical procedure, but without the exposure to a magnetic field (Mag−group).

Results: Two days after delivery the animals were imaged using a bioluminescence detection system with the injection of luciferin. The signal emitted from the stented arterial segment due to the luciferase transgene was an order of magnitude higher in the animals that received cells in the presence of a magnetic field (Mag+group). The Quantitative data shown in FIGS. 11B and 11D are expressed as means±se. Student's t-test was used to determine the statistical significance. Differences were termed significant at P<0.05.

Conclusion: The functionality of magnetically targeted cells to stent surfaces was demonstrated by a robust adenoviral-transgene expression 2 days post treatment. This demonstrates magnetic targeting of genetically modified cells as a therapeutic method for vascular applications of implantable devices.

Having described the invention, we now claim the following and their equivalents.

1. A magnetically assisted therapeutic system comprising:
(a) a therapeutic formulation administered to a mammalian subject by peripheral intravenous administration, wherein the therapeutic formulation comprises particles of a magnetic or magnetizable material that carry a therapeutic agent;
(b) an implantable device implanted in a vascular system of a mammalian subject, the implanted implantable device comprising a biocompatible magnetic or magnetizable material; and
(c) a retrieval system having a magnetic or magnetizable mesh operably connected to the mammalian subject.

2. The magnetically assisted therapeutic system of claim 1, further comprising a magnetic field generator for generating a directable magnetic field gradient in proximity of the implanted implantable device, wherein the directable magnetic field gradient directs the magnetic or magnetizable material in proximity to the implanted device.

3. The magnetically assisted therapeutic system of claim 1, further comprising a magnetic field generator for generating a directable magnetic field gradient in proximity to the magnetic or magnetizable mesh, wherein the directable magnetic field gradient directs a portion of the magnetic or magnetizable material that is not delivered to the implanted device to the magnetic or magnetizable mesh.

4. The magnetically assisted therapeutic system of claim 1 wherein the magnetic or magnetizable mesh is configured as a filter within a cardiovascular circulation circuit of the retrieval system that promotes the apheresis.

5. The magnetically assisted therapeutic system of claim 1 wherein the retrieval system is configured to prevent the magnetic or magnetizable material from accumulating in a reticulo-endothelial system of the mammalian subject.

6. The magnetically assisted therapeutic system of claim 1 wherein the surface of the particles is modified such that the therapeutic formulation remains in circulation for a number of cardiac cycles of the mammalian subject.

7. The magnetically assisted therapeutic system of claim 6 wherein the surface of the particles is modified with a biocompatible hydrophilic polymer.

8. The magnetically assisted therapeutic system of claim 6 wherein the surface of the particles is modified with serum albumin.

9. The magnetically assisted therapeutic system of claim 1 wherein the implanted implantable device is a stent.

10. A method for administering a therapeutic agent, the method comprising the steps of:
(a) intravenously administering a therapeutic formulation to a vascular system of a mammalian subject, wherein the therapeutic formulation comprises particles of a biocompatible magnetic or magnetizable material that carry the therapeutic agent;
(b) delivering a portion of the therapeutic formulation to the proximity of an implantable device implanted in the vascular system in the mammalian subject by externally generating a magnetic field gradient on the implantable device, wherein the implantable device comprises a biocompatible magnetic or magnetizable material; and
(c) removing a portion of the therapeutic formulation that is not delivered to the proximity of the implantable device from the vascular system.

11. The method of claim 10 wherein the therapeutic formulation is peripherally injected from a site of the implantable device.

12. The method of claim 10 wherein the therapeutic formulation is locally injected at a site of the implantable device.

13. The method of any claim 10 wherein the implantable device is intravascularly implanted in the mammalian subject.

14. The method of any claim 10 wherein step (c) further comprises the steps of:
(c1) promoting apheresis of the therapeutic formulation during cardiovascular circulation to direct the portion of the therapeutic formulation to a magnetized or magnetizable mesh; and
(c2) delivering the directed portion of the therapeutic formulation to the magnetized or magnetizable mesh by externally generating a further magnetic field gradient on the magnetized or magnetizable mesh.

15. The method claim 10 wherein step (b) is performed for a predetermined duration of time.

16. The method of claim 10 further comprising repeating steps (a) to (c).
